(19) United States
(12) Patent Application Publication
(10) Pub. No.: US 2005/0138681 A1
(43) Pub. Date: Jun. 23, 2005

(54) Soybean containing high levels of free amino acids

(75) Inventors: Masakazu Takahashi, Kumamoto (JP); Masao Ishimoto, Hiroshima (JP); Makita Hajika, Ibaraki (JP); Ryoichi Matsunaga, Ibaraki (JP); Kunihiko Komatsu, Kumamoto (JP); Keisuke Kitamura, Hokkaido (JP); Kazuhiro Yagasaki, Nagano (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Inc Admin Agcy Natl Agric and Bio-oriented Res Org, Tsukuba-shi (JP)

(21) Appl. No.: 10/812,916

(22) Filed: Mar. 31, 2004

(30) Foreign Application Priority Data
Sep. 30, 2003 (JP) 2003-342020

(51) Int. Cl. A01H 1/00; C12N 15/82; A01H 5/00

(52) U.S. Cl. 800/278; 800/312

(57) ABSTRACT

The present invention relates to a soybean, which can be easily processed into a food containing free amino acids and has a high total free amino acid content in the seeds thereof, and a method of producing such soybean. By providing a soybean characterized in that it genetically lacks all subunits of β-conglycinin and glycinin, or has only A₃B₄, which is the subunit of glynicin among the subunits of β-conglycinin and glycinin, a soybean having a total free amino content in the seeds thereof, which is greater than those of general soybean varieties, is provided.

### BACKGROUND OF THE INVENTION

1. Technical Field

The present invention relates to a soybean, the seeds of which contain free amino acids at a higher concentration than those of a conventional soybean variety, and a method of producing the same.

2. Background Art

It is well known that in the seeds of soybeans, which are legumes, large quantities of storage proteins are accumulated. Most amino acids contained in the seed are stored in the linked form, that is, stored as components of such as storage proteins or enzyme proteins. Hence, not many of amino acids are present in a free state within soybean seeds. Thus a high concentration of free amino acids cannot be obtained from the seeds unless proteins are hydrolyzed by any of enzyme methods, a chemical preparation methods or the like, which are known.

Soybean seeds contain β-conglycinin and glycinin as major storage proteins, which account for approximately 70% of the total proteins. Therefore, the properties of soybean proteins are mostly reflections of the characteristics of these two components. To improve the properties of soybean storage proteins, it is effective to change β-conglycinin and glycinin contents. For this purpose, some types of soybeans have been produced wherein the subunits of both proteins are genetically deleted or reduced. For example, one finding that has been obtained thus far (e.g., Ogawa et al. 1989) is that genetic deletion of α and α′ and the reduction of β subunit content of β-conglycinin complementarily increase the glycinin content, so that the content of sulfur-containing amino acids (cystine and methionine), which limits the nutritional value of soybean proteins, can be increased.

Also regarding five subunits, A₁ₐB₂, A₂B₁ₐ, A_{1b}B_{1b}, A₅A₄B₃ and A₃B₄, composing glycinin, the structure and expression mechanism of Gy₁, Gy₂, Gy₃, Gy₄ and Gy₅ genes for the synthesis of these subunits have been elucidated (e.g., Nielsen et al. 1988). Moreover, a subunit-deficient soybean mutant line, which is a type of a line simultaneously deficient in A₁ₐB₂, A₂B₁ₐ and A_{1b}B_{1b} (Group I), a type of a line that is deficient in A₅A₄B₃ (Group IIa), and a type of a line that is deficient in A₃B₄ (Group IIb) have been produced. Thus, it is known that these types of subunit-deficient mutant lines are each governed by a recessive gene (e.g., Yagasaki et al. 1996). This makes it possible to produce a soybean that genetically lacks multiple subunits of glycinin by crossing the above mutant lines as parents. Thus, the relationship between glycinin content and suitability for processing, particularly suitability for processing tofu, has come to be examined (Yagasaki et al. 2000).

As described above, past studies on proteins and amino acids of soybean seeds have been intended exclusively for storage proteins. There have been few reports concerning free amino acids in soybean seeds. Furthermore, there have been no ideas about increasing the level of free amino acids in soybean seeds by utilizing storage protein-deficient soybeans.

However, recently in view of nutritional physiology, food studies and the like, free amino acid ingestion is believed to be desirable for, for example, recovery from fatigue and/or improvement in fat-burning efficiency. Various functional foods or supplements containing free amino acids have been developed.

The soybean is a plant that is cultivated as a greatly useful edible crop for the human race in great many countries and areas. The soybean seeds are utilized as vegetable foods rich in oil and protein content in a variety of processed foods.

Particularly in recent years, the fact that the ingestion of a variety of free amino acids has a good effect on human health is now widely known. Thus, sports supplements and diet supplements utilizing an effect of recovery from fatigue, an in vivo fat-burning effect and the like due to free amino acids are now broadly marketed. Arginine, which is contained particularly richly in the soybean of the present invention, has been reported that arginine ingestion helps the disruption of malignant tumor cells (e.g., Park et al. 1991), and is useful for improving human immune functions (e.g., Kirk et al. 1993) and enhancing the secretion of growth hormones (e.g., Kreider et al. 1993). Glutamine has also been reported to be useful for enhancing muscle force (e.g., Rennie 1996), improving immune functions (e.g., Newsholme and Calder 1997) and the like. Various health functionalities of free amino acids have been scientifically examined.

### SUMMARY OF THE INVENTION

Hence, if a soybean with high total free amino acid content in the seeds thereof can be produced, functional soybean-processed foods having a high total free amino acid content can be obtained using such seeds as raw materials.

An object of the present invention is to provide a soybean, wherein amino acids produced as the initial product are accumulated with their free state unchanged at a high concentration in the seed thereof, by genetically suppressing the ability of biosynthesizing major storage proteins in the soybean seed. Another object of the present invention is a method of producing the same. Moreover, according to this method, a soybean with high free amino acid content is provided as a raw material for functional processed foods that contain free amino acids possessing a variety of health functionalities.

To achieve the above objectives, we intensively studied and thus, for the first time in the world, discovered β-conglycinin-deficient soyabeen, Glycine soja QT2 (deposited on May 8, 2003, with the International Patent Organism Depositary at the National Institute of Advanced Industrial Science and Technology (Central 6, 1-1-1 Higashi, Tsukuba, Ibaraki, Japan) under accession No. FERM BP-8376), which lacks all α, α′ and β subunits (Hajika et al. 1996). As a result of further studies, we discovered that the β-conglycinin deficiency is governed by a dominant gene that is a single factor, by crossing β-conglycinin-deficient Glycine soja QT2 with a conventional soybean variety having β-conglycinin, and calculating the segregation ratio of F₁ and F₂ plant groups. We named the gene Scg (Hajika et al.1998).

Furthermore, we further back-crossed β-conglycinin-deficient Glycine soja with conventional soybean varieties, thereby producing Kyukei 305, which is a β-conglycinin-deficient soybean having agricultural properties (such as flowering, maturation and growth state) equivalent to those of conventional soybean varieties (Takahashi et al. 2000).

We further crossed a soybean (Yagasakai et al. 1996) genetically lacking all the major subunits of glycinin, A₁ₐB₂, A₂B₁ₐ, A_{1b}B_{1b}, A₅A₄B₃ and A₃B₄ with another conventional soybean (Enrei), thereby producing EnB1, which is a glycinin-deficient soybean variety having partially improved agricultural properties (deposited on May 8, 2003 with International Patent Organism Depositary at the National Institute of Advanced Industrial Science and Technology (Central 6, 1-1-1 Higashi, Tsukuba, Ibaraki, Japan) under accession No. FERM BP-8377).

Furthermore, we discovered for the first time in the world that a soybean genetically lacking β-conglycinin and glycinin, which account for approximately 70% of storage proteins, can be produced by crossing a β-conglycinin-deficient soybean with a glycinin-deficient soybean (Uematsu et al., 1999).

We completed the present invention by selecting soybeans genetically lacking β-conglycinin and glycinin simultaneously from the filial generations resulting from the crossing of the β-conglycinin-deficient soybean with the glycinin-deficient soybean, and then finding particularly increased total free amino acid content in the seeds.

The present invention relates to the following embodiments: p0 1. A soybean, having total free amino acid content in the seed thereof that is higher than the content in the seeds of any of Fukuyutaka and Tachiyutaka having all subunits of β-conglycinin and glycinin, Enrei lacking only A₅A₄B₃ subunit of glycinin, Kyukei 305 lacking all subunits of β-conglycinin and EnB1 lacking all subunits of glycinin that are cultivated under similar conditions.
2. The soybean of claim 1, having total free amino acid content in the seed thereof that is at least 2 times or more greater than the content in the seeds of any of Fukuyutaka and Tachiyutaka having all subunits of β-conglycinin and glycinin, Enrei lacking only A₅A₄B₃ subunit of glycinin, Kyukei 305 lacking all subunits of β-conglycinin, and EnB1 lacking all subunits of glycinin that are cultivated under similar conditions.
3. The soybean of claim 1, wherein the total free amino acid content in the seed thereof is 8 mg or more per gram dry weight of the seed.
4. The soybean of claim 1, wherein the content of at least one of free amino acids selected from the group consisting of arginine, asparaine, histidine and glutamine among each of free amino acids contained in the seed is greater than that contained in the seed of any of Fukuyutaka and Tachiyutaka having all subunits of β-conglycinin and glycinin, Enrei lacking only A₅A₄B₃ subunit of glycinin, Kyukei 305 lacking all subunits of β-conglycinin and EnB1 lacking all subunits of glycinin that are cultivated under similar conditions.
5. The soybean of claim 4, wherein the contents of each free amino acids of arginine, asparagine, histidine and glutarine among each of free amino acids contained in the seed are each greater than the contents thereof in the seeds of any of Fukuyutaka and Tachiyutaka having all subunits of β-conglycinin and glycinin, Enrei lacking only A₅A₄B₃ subunit of glycinin, Kyukei 305 lacking all subunits of β-conglycinin and EnB1 lacking all subunits of glycinin wherein they are cultivated under similar conditions.
6. The soybean of claim 1, genetically lacking at least α, α′ and β subunits of β-conglycinin, and A₁ₐB₂, A₂B₁ₐ, A_{1b}B_{1b} and A₅A₄B₃ subunits of glycinin.
7. The soybean of claim 6, genetically lacking all subunits of β-conglycinin and glycinin.
8. A method of producing the soybean of claim 6, comprising either step of crossing a soybean lacking one or more subunits selected from the group consisting of α, α′ and β subunits of β-conglycinin, and A₁ₐB₂, A₂B₁ₐ, A_{1b}B_{1b} and A₅A₄B₃ subunits of glycinin with a soybean lacking all the subunits contained in the above soybean among the subunits in the above group, or step of crossing a soybean lacking all of the above subunits with a soybean having all of or some of these subunits, wherein at least one of the two soybeans to be crossed herein has the A₃B₄ subunit of glycinin.
9. The method of producing the soybean of claim 7, comprising either step of crossing a soybean lacking one or more subunits selected from the group consisting of α, α′ and β subunits of β-conglycinin and A₁ₐB₂, A₂B₁ₐ, A_{1b}B_{1b}, A₅A₄B₃ and A₃B₄ subunits of glycinin with a soybean lacking all the subunits of the soybean among the above subunits, or step of crossing a soybean lacking all of the subunits in the above group with a soybean having all of or some of these subunits.
10. The method of claim 8 or 9, comprising a step of crossing Kyukei 305 lacking all subunits of β-conglycinin with EnB1 lacking all subunits of glycinin.
11. The method of any one of claims 8 to 10, further comprising a step of selecting a line having only the A₃B₄ subunit of glycinin among the subunits of β-conglycinin and glycinin, or a line lacking all subunits of β-conglycinin and glycinin following the step of crossing.
12. A functional food, which is produced using the soybean seed of any one of claims 1 to 7 as a raw material, and wherein the total free amino acid content is increased.
13. A method of producing a functional food wherein the total free amino acid content is increased, using the soybean seed of any one of claims 1 to 7 as a raw material.

### DETAILED DESCRIPTION OF THE INVENTION

The term “total free amino acid content” in this specification indicates the total sum of each amino acid that is not incorporated in proteins, and is thus in a free state in each soybean seed. A method for determining the quantity of each free amino acid is known in the art. For example, a free amino acid extract extracted from pulverized seeds using a solvent such as ethanol may be subjected to amino acid analysis. All 20 types of amino acids existing in nature may be respectively determined, so as to calculate the total sum.

Even under different environmental conditions for cultivation, such as pot cultivation (medium: a mixture of culture soil and humus soil at a 2:1 ratio) within a heating glasshouse (adjusted to an average temperature of 25° C., and having a natural day length) from winter to spring (December 1998 to March 1999), general farmland (black volcanic ash soil, with an average temperature of 24.3° C., average maximum temperature of 29.3° C., average minimum temperature of 19.7° C. and precipitation of 453.0 mm) from summer to fall (July to October 2000), and general farmland (black volcanic ash soil, with an average temperature of 24.1° C., average maximum temperature of 29.2° C., average minimum temperature of 19.4° C. and precipitation of 682.5 mm) from summer to fall (July 2001 to October 2001), the soybean of the present invention was found to have a total free amino acid content in the seeds thereof that was greater, preferably 2 times or more, further preferably 3 times or more, and most preferably 4 times or more greater than that of any of soybeans selected from the group consisting of a soybean such as Fukuyutaka and Tachiyutaka that are not deficient in any subunits of β-conglycinin and glycinin, the major soybean storage proteins, and Enrei that genetically lacks A₅A₄B₃ subunit of glycinin, a soybean such as Kyukei 305 that genetically lacks all subunits of β-conglycinin, and a soybean such as EnB1 that genetically lacks all subunits of glycinin, when they were cultivated under similar conditions. In the case of cultivation on normal farmland, the total content of all free amino acids in the above cultivated soybean seeds differs depending on type and/or cultivation conditions, and is often within the range between 1 and 3 mg per gram of normal seeds. The total amino acid content of the soybean of the present invention is 5 mg or more, preferably 8 mg or more, further preferably 9 mg or more, and even further preferably 10 mg or more, and most preferably 30 mg or more per gram of the seeds.

Furthermore, when the content of each amino acid is examined, among free amino acids, arginine, asparagine, histidine and glutamine contents are increased in the soybean of the present invention compared with other conventional soybean varieties cultivated under similar conditions. Though each free amino acid content differs depending on the variety or line among conventional soybean varieties, for example, compared with any variety or line selected from the group consisting of Fukuyutaka, Tachiyutaka, Enrei, Kyukei 305 and EnB1 cultivated under similar conditions, asparagine content and histidine contents are 2 times or more, glutamine content is 5 times or more, and arginine content is 8 times or more greater in the soybean of the present invention.

One embodiment of the present invention is a soybean having only the A₃B₄ subunit of glycinin among subunits composing β-conglycinin and glycinin. Such a soybean can be produced by a method comprising either a step of crossing a soybean genetically lacking one or more subunits selected from the group consisting of α, α′ and β subunits of β-conglycinin, and A₁ₐB₂, A₂B₁ₐ, A_{1b}B_{1b} and A₅A₄B₃ subunits of glycinin with a soybean genetically lacking all the subunits contained in the above soybean among subunits in the above group; or a step of crossing a soybean genetically lacking all the above subunits with a soybean having all of or some of them. Here, at least one of two soybeans to be crossed may have A₃B₄ subunit of glycinin. Specifically, when at least one of two soybeans to be crossed genetically lacks all subunits of β-conglycinin and all of A₁ₐB₂, A₂B₁ₐ, A_{1b}B_{1b} and A₅A₄B₃ subunits of glycinin, and at least one of two soybeans to be crossed can express the A₃B₄ subunit of glycinin, a soybean having only A₃B₄ subunit of glycinin among subunits composing β-conglycinin and glycinin can be produced by crossing these soybeans.

Furthermore, the soybean of the present invention may be a soybean genetically lacking all subunits composing β-conglycinin and glycinin. Such a soybean can be produced by crossing a soybean genetically lacking one or more subunits selected from the group consisting of α, α′ and β subunits of β-conglycinin, and A₁ₐB₂, A₂B₁ₐ, A_{1b}B_{1b}, A₅A₄B₃ and A₃B₄ subunits of glycinin with a soybean genetically lacking all the subunits of the soybean among the above subunits; or crossing a soybean genetically lacking all the above subunits with a soybean having all of or some of these subunits. For example, such a soybean can be obtained by crossing Kyukei 305 genetically lacking all subunits of β-conglycinin with EnB1 genetically lacking all subunits of glycinin, or crossing Fukuyutaka having all subunits of β-conglycinin and glycinin with a soybean genetically lacking all subunits of β-conglycinin and glycinin.

Crossing of soybeans can be performed by a method known in the art.

Soybeans genetically lacking at least one subunit, all subunits other than the A₃B₄ subunit, or all subunits among the subunits composing β-conglycinin and glycinin, may be selected from those of the obtained filial generation. Determination of whether or not a soybean lacks a subunit(s) for this selection can be easily performed by persons skilled in the art For example, a cotyledon portion of a seed obtained from the filial generation is scraped off, and then determination can be performed by an SDS-polyacrylamide gel electrophoresis method according to the method of Kitamura et al., (1984).

Next, a soybean line lacking the subunits of β-conglycinin and glycinin can be genetically fixed. A soybean line (hereinafter referred to as QF2) genetically lacking all subunits composing β-conglycinin and glycinin, or a soybean line (hereinafter referred to as QF3) genetically lacking all subunits of β-conglycinin and groups I (A₁ₐB₂, A₂B₁ₐ and A_{1b}B_{1b}) and IIa (A₅A₄B₃) subunits of glycinin can also be selected and fixed. These methods are known by persons skilled in the art.

Therefore, the present invention also encompasses these methods of producing the soybean of the present invention.

The soybean of the present invention that is produced by the above-mentioned method possesses agricultural properties equivalent to those of general and conventional soybean varieties, exhibits normal vegetative growth in general field cultivation, and can produce normal seeds. Thus the soybean of the present invention actually causes no problems as a cultivated plant.

When the total free amino acid content of a soybean genetically lacking only all subunits of β-conglycinin is measured as a reference to it with the soybean of the present invention, no increase is observed in free amino acids, compared with the case of a conventional soybean variety or a soybean that is not deficient in any subunits of β-conglycinin and glycinin. In contrast, when the total free amino acid content of a soybean genetically lacking only all subunits of glycinin is compared with that of a conventional soybean variety, the contents of some free amino acids are found to be larger, but such larger quantities are obviously lower than those of QF2 and QF3 soybeans. Causing genetic deficiency in some subunits of glycinin in addition to all subunits of β-conglycinin is also effective in terms of increasing the free amino acid content of the seed. However, the degree of the increase is inferior to the cases of QF2 and QF3. Moreover, even in the case of a soybean genetically lacking most of β-conglycinin, that is, a soybean, which is incompletely deficient in β-conglycinin and deficient in glycinin and is obtained from the filial generation resulting from the crossing of a β-conglycinin-incompletely-deficient soybean (which genetically lacks α and α′ subunits, and the level of β subunit synthesized is reduced) with a glycinin-deficient soybean, the total free amino acid content in the seed is as same as that of the glycinin-deficient soybean. Therefore, the soybean of the present invention genetically lacking at least all subunits of β-conglycinin and groups I (A₁ₐB₂, A₂B₁ₐ and A_{1b}B_{1b}) and IIa (A₅A₄B₃) subunits of glycinin, among all subunits of β-conglycinin and glycinin, specifically, the soybean having only group IIb (A₃B₄) subunit of glycinin, among subunits of β-conglycinin and glycinin, or the soybean genetically lacking all subunits of β-conglycinin and glycinin is advantageous as a food crop in that the total free amino acid content is particularly high compared with other types.

Furthermore, the present invention also encompasses processed foods produced using the soybean seeds of the present invention as a raw material. The processed foods include various soybean-processed foods including soybean milk (including modified soy milk and soy milk drink), tofu and the like. Since the total free amino acid contents are high in the soybean seeds used as raw materials, these processed foods have higher total free amino acid contents, particularly higher free arginine, asparagine, histidine and glutamine contents compared with those of processed foods that are produced by similar methods using conventional soybean seeds as raw materials. Thus, by the use of the soybean of the present invention, soybean-processed foods with high total free amino acid contents without treatment such as addition of free amino acids can be obtained. Accordingly, the processed food of the present invention enables the efficient absorption of amino acids and is nutritionally superior.

Effect of the Invention

According to the present invention, a soybean line having total free amino acid content in the seeds thereof that is greater than the content found in the seeds of conventional soybean varieties or lines is produced. This soybean possesses agricultural properties that are appropriate for agricultural application. Furthermore, the soybean produced by the present invention has a total free amino acid content which is 3 to 5 times or more greater than those of conventional general soybean varieties. In particular, the soybean of the present invention has high free arginine, asparagine, histidine and glutamine contents. Accordingly, when a human ingests the soybean obtained by the present invention instead of a conventional soybean, the human becomes able to efficiently absorb a larger quantity of free amino acids directly in the body.

Hence, the soybean obtained by the present invention possesses extremely high usefulness as a food material for functional foods with high contents of free amino acids, unlike conventional soybean varieties.

### EXAMPLES

The present invention will be described more specifically by the following examples. These examples are not intended to limit the scope of the present invention.

### Example 1

First a method of producing soybeans, which have high levels of free amino acids and genetically lack all subunits of β-conglycinin and glycinin, was verified. Kyukei 305 genetically lacking all subunits of β-conglycinin and EnB1 genetically lacking all subunits of glycinin were grown for approximately 40 to 50 days by pot soil cultivation (black volcanic ash soil) in a heating glasshouse (Nishi-goshi machi, Kumamoto, Japan; adjusted to a minimum temperature of 22° C. and a maximum temperature of 32° C.; for 1 month after sowing, lightning was performed everyday with fluorescent lamps twice a day (4 p.m. to 9 p.m. and 3 a.m. to 8 a.m. in the next morning) to give a day length of 18 hours; and after this period, plants were grown under natural day length conditions) during a period from December 1997 to March 1998. Then the plants were crossed, thereby obtaining F₁ seeds (total days of growth: approximately 95 days). Next, F₁ seeds were sown in general farmland (Nishi-goshi machi, Kumamoto, Japan, black volcanic ash soil) in July, 1998. Then the germinated F₁ plants were grown to maturing in the middle of October, 1998 (with an average temperature of 24.9° C., average maximum temperature of 30.4° C., average minimum temperature of 20.3° C. and total precipitation of 301.5 mm), thereby obtaining F₂ seeds. The cotyledon portions (approximately 10 mg) of the thus obtained F₂ seeds were scraped off, and then the presence or the absence of each subunit group of β-conglycinin and glycinin was determined by the SDS-polyacrylamide gel electrophoresis method according to the method of Kitamura et al (1984). Accordingly, F₂ seeds genetically lacking all subunits of β-conglycinin and glycinin were selected. Furthermore, F₂ seeds genetically lacking all subunits of β-conglycinin and glycinin were sown in pots (filled with a mixture of culture soil and humus soil at a 2:1 ratio) in a heating glasshouse (Fukuyama-shi, Hiroshima, Japan; adjusted to an average temperature of 25° C., and having a natural day length) in December 1998. After the germinated F₂ plants were grown, F₃ seeds were obtained in March 1999. Approximately 30 grains of the F₃ seeds obtained from each plant were analyzed by the SDS-polyacrylamide gel electrophoresis method, thereby selecting an F₃ line whose seeds were all genetically deficient in all subunits of β-conglycinin and glycinin. Specifically, lines (QF2F₃-1, QF2F₃-2 and QF2F₃-3) wherein deficiency in all subunits of β-conglycinin and glycinin had been genetically fixed were selected. Of these 3 lines, seeds of 2 lines (QF2F₃-1 and QF2F₃-2) were pulverized, approximately 500 mg of the pulverized product was precisely weighed, and was then used for determining free amino acids. In addition, for comparison, a conventional soybean variety (Tachiyutaka) having all subunits composing β-conglycinin and glycinin, a β-conglycinin-deficient soybean (Kyukei 305) and a glycinin-deficient soybean (EnB1), which had been used as parents for crossing were cultivated under the same conditions employed for F₂ plants, and then their seeds were obtained. These seeds were compared by pulverizing them, precisely weighing approximately 500 mg of each pulverized product and then determining amounts of free amino acids.

For determination of amounts of free amino acids, the above-precisely weighed and pulverized seeds (10 mg in weight of the seeds as a sample) were mixed with 200 μl of 75% ethanol in vitro. After 2 minutes of shaking at room temperature, centrifugation was performed at 5,000 g for 5 minutes, and then the supernatant was obtained as a free amino acid extract. Furthermore, 200 μl of 75% ethanol was added to the precipitated seed residue per 10 mg in weight of the pulverized seeds as a sample, and then again, a free amino acid extract was obtained similarly. After mixing these two extracts together, a one-twentieth volume thereof was sampled, subjected to centrifugation at 15,000 g for 10 minutes to remove residue, and then diluted 5-fold with distilled water. The diluted solution was centrifuged again at 6,000 g for 50 minutes. Using 1 ml of the thus obtained supernatant, the contents of 20 types of amino acids were measured using a Pico Tag amino acid analysis system, and then the total sum was calculated as the total free amino acid content. Table 1 shows the results.

TABLE 1
Results-1 Analysis of free amino acids in soybean seeds

| Variety or | | Kyukei | | | |
|---|---|---|---|---|---|
| line name | Tachiyutaka | 305 | EnB1 | QF2F₃-1 | QF2F₃-2 |
| Presence or | + | − | + | − | − |
| absence | | | | | |
| of β-conglycinin | | | | | |
| Presence or | + | + | − | − | − |
| absence of | | | | | |
| glycinin | | | | | |
| Free amino | 4.18 | 4.64 | 4.86 | 25.48 | 22.55 |
| acid content | | | | | |
| (mg/g seed) | | | | | |

Note 1)
Tachiyutaka is a conventional soybean variety.
Note 2)
Kyukei 305 and EnB1 are mother plant and father plant of QF2F₃-1 and QF2F₃-2.

As is clear from Table 1, total free amino acid contents of β-conglycinin-deficient soybean (Kyukei 305) and glycinin-deficient soybean (EnB1) were higher than that of the conventional soybean variety (Tachiyutaka). However, the difference was small. The total free amino acid content of glynicin-deficient soybean (EnB1), which was the highest among the results, was around 1.16 times greater than that of the conventional soybean variety (Tachiyutaka).

Total free amino acid contents in the seeds of QF2F₃-1 and QF2F₃-2 genetically lacking all subunits of β-conglycinin and glycinin were 25.5 and 22.6 mg/g, respectively, which were 5 times or more greater than that of the conventional soybean variety (Tachiyutaka). By the above examples, a method of producing soybeans with high levels of free amino acids, which genetically lack all subunits of β-conglycinin and glycinin, could be verified.

### Example 2

Next, all lines that can be produced from the combinations for the crossing of Kyukei 305 (β-conglycinin-deficient soybean) with EnB1 (glycinin-deficient soybean) performed in Example 1, that is, 16 types of lines differing in their subunit compositions of β-conglycinin and glycinin, were produced. Then, it was verified whether soybeans, which were equivalent to other soybeans with high levels of free amino acids (QF2, obtainable by causing genetic deficiency in all subunits of β-conglycinin and glycinin), were obtained from these lines. Specifically, F₂ seed population that had remained after the selection of the F₂ seeds (QF2F₂-1, QF2F₂-2 and QF2F₂-3) genetically lacking all subunits of β-conglycinin and glycinin from the F₂ population obtained in Example 1 was grown in December 1999 in a heating glasshouse (Nishi-goshi machi, Kumamoto, Japan; adjusted to a minimum temperature of 22° C. and a maximum temperature of 32° C.; for 1 month after sowing, lightning was performed everyday with fluorescent lamps twice a day (4 p.m. to 9 p.m., and 3 a.m. to 8 a.m. in the next morning) to give a day length of 18 hours, and after this period, plants were grown under natural day length conditions.) Thus, F₃ seeds thereof were obtained in March 2000. The thus obtained F₃ seeds were subjected to the SDS-polyacrylamide gel electrophoresis method. 16 types of soybean seeds differing in their subunit compositions of β-conglycinin and glycinin shown in Table 2, specifically, each 10 to 20 grains of F₃ soybean seeds having all subunits of β-conglycinin and glycinin, F₃ soybean seeds genetically and partially lacking subunits of β-conglycinin and glycinin, and F₃ soybean seeds genetically lacking all subunits of β-conglycinin and glycinin were prepared. The 16 thus obtained types of F₃ soybean seeds were cultivated during a period from July to October 2000 (with an average temperature of 24.3° C., average maximum temperature of 29.3° C., average minimum temperature of 19.7° C., and precipitation of 453.0 mm) in outdoor general farmland (Nishi-goshi machi, Kumamoto, Japan, black volcanic ash soil). Regarding approximately 15 grains of the F₄ seeds obtained from each F₃ plant, F₃ plants that had produced only F₄ seeds whose subunit compositions of β-conglycinin and glycinin were the same as those of the parent F₃ plant and fixed were selected. Thus, 16 types of F₄ lines differing in their subunit compositions of β-conglycinin and glycinin were obtained as shown in Table 2. The seeds of 16 types of F₄ lines were pulverized and approximately 500 mg of the pulverized product of each F₄ line was precisely weighed. Then, free amino acid contents were analyzed by the method similar to that given in Example 1, and then the total quantity was calculated. Table 2 shows the results.

TABLE 2
Results-2 Analysis of free amino acids in soybean seeds

| | | | F₄ line | | | | | | | |
|---|---|---|---|---|---|---|---|---|---|---|
| | | | 1 | 2 | 3 | 4 | 5 | 6 | 7 | 8 |
| β-conglycinin | α | | + | + | + | + | + | + | + | + |
| subunit | α′ | | + | + | + | + | + | + | + | + |
| | β | | + | + | + | + | + | + | + | + |
| glycinin subunit | I | A₁ₐB₂, A₂B₁ₐ, A_{1b}B_{1b} | + | + | − | − | − | + | + | − |
| | IIa | A₅A₄B₃ | + | − | + | − | + | − | + | − |
| | IIb | A₃B₄ | + | − | − | + | + | + | − | − |
| free amino acid | Asp | aspartic acid | 0.388 | 0.456 | 0.465 | 0.365 | 0.270 | 0.260 | 0.369 | 0.613 |
| mg/g DW | Glu | glutamic acid | 0.237 | 0.500 | 0.415 | 0.457 | 0.243 | 0.207 | 0.206 | 0.441 |
| | Ser | serine | 0.021 | 0.021 | 0.028 | 0.025 | 0.021 | 0.024 | 0.015 | 0.030 |
| | Asn | asparagine | 0.107 | 0.152 | 1.245 | 0.344 | 0.402 | 0.397 | 0.035 | 0.622 |
| | Gly | glycine | 0.013 | 0.018 | 0.024 | 0.018 | 0.016 | 0.015 | 0.010 | 0.020 |
| | Gln | glutamine | 0.006 | 0.008 | 0.005 | 0.010 | 0.007 | 0.005 | 0.005 | 0.008 |
| | His | histidine | 0.072 | 0.165 | 0.236 | 0.127 | 0.066 | 0.086 | 0.018 | 0.178 |
| | Thr | threonine | 0.023 | 0.022 | 0.027 | 0.017 | 0.019 | 0.016 | 0.009 | 0.013 |
| | Ala | alanine | 0.053 | 0.112 | 0.089 | 0.089 | 0.061 | 0.057 | 0.046 | 0.102 |
| | Arg | arginine | 0.266 | 0.183 | 0.462 | 0.542 | 0.281 | 0.317 | 0.101 | 0.691 |
| | Pro | proline | 0.027 | 0.041 | 0.040 | 0.038 | 0.032 | 0.035 | 0.033 | 0.032 |
| | Tyr | tyrosine | 0.145 | 0.088 | 0.098 | 0.078 | 0.088 | 0.077 | 0.131 | 0.145 |
| | Val | valine | 0.046 | 0.052 | 0.061 | 0.049 | 0.045 | 0.043 | 0.044 | 0.065 |
| | Met | methionine | 0.012 | 0.012 | 0.011 | 0.014 | 0.009 | 0.008 | 0.015 | 0.020 |
| | Cys | cysteine | 0.002 | 0.002 | 0.002 | 0.002 | 0.002 | 0.002 | 0.002 | 0.003 |
| | Ile | isoleucine | 0.014 | 0.022 | 0.020 | 0.021 | 0.017 | 0.014 | 0.019 | 0.026 |
| | Leu | leucine | 0.024 | 0.033 | 0.019 | 0.027 | 0.016 | 0.023 | 0.024 | 0.023 |
| | Phe | phenylalanine | 0.064 | 0.062 | 0.036 | 0.035 | 0.047 | 0.059 | 0.066 | 0.052 |
| | Trp | tryptophan | 0.130 | 0.139 | 0.182 | 0.220 | 0.141 | 0.144 | 0.103 | 0.228 |
| | Lys | lysine | 0.022 | 0.026 | 0.027 | 0.024 | 0.025 | 0.024 | 0.024 | 0.032 |
| | total | | 1.670 | 2.112 | 3.490 | 2.502 | 1.809 | 1.813 | 1.274 | 3.344 |
| | | | F_{4 line} | | | | | | | |
| | | | 9 | 10 | 11 | 12 | 13 | 14 | 15 | 16 |
| β-conglycinin | α | | − | − | − | − | − | − | − | − |
| subunit | α′ | | − | − | − | − | − | − | − | − |
| | β | | − | − | − | − | − | − | − | − |
| glycinin subunit | I | A₁ₐB₂, A₂B₁ₐ, A_{1b}B_{1b} | + | + | − | − | − | + | + | − |
| | IIa | A₅A₄B₃ | + | − | + | − | + | − | + | − |
| | IIb | A₃B₄ | + | − | − | + | + | + | − | − |
| free amino acid | Asp | aspartic acid | 0.420 | 0.751 | 0.549 | 1.106 | 0.650 | 0.582 | 0.706 | 1.105 |
| mg/g DW | Glu | glutamic acid | 0.306 | 0.639 | 0.575 | 0.843 | 0.436 | 0.490 | 0.406 | 0.980 |
| | Ser | serine | 0.021 | 0.054 | 0.049 | 0.087 | 0.031 | 0.050 | 0.035 | 0.080 |
| | Asn | asparagine | 0.153 | 1.325 | 0.915 | 2.929 | 0.528 | 1.218 | 1.267 | 1.587 |
| | Gly | glycine | 0.012 | 0.024 | 0.074 | 0.051 | 0.020 | 0.025 | 0.022 | 0.033 |
| | Gln | glutamine | 0.007 | 0.016 | 0.014 | 0.047 | 0.012 | 0.017 | 0.014 | 0.060 |
| | His | histidine | 0.051 | 0.405 | 0.278 | 0.699 | 0.109 | 0.439 | 0.232 | 0.405 |
| | Thr | threonine | 0.013 | 0.024 | 0.028 | 0.057 | 0.025 | 0.024 | 0.019 | 0.037 |
| | Ala | alanine | 0.051 | 0.108 | 0.140 | 0.149 | 0.086 | 0.094 | 0.088 | 0.116 |
| | Arg | arginine | 0.253 | 1.965 | 1.656 | 3.800 | 0.586 | 1.678 | 2.000 | 4.359 |
| | Pro | proline | 0.029 | 0.033 | 0.055 | 0.044 | 0.043 | 0.038 | 0.024 | 0.032 |
| | Tyr | tyrosine | 0.117 | 0.121 | 0.125 | 0.137 | 0.114 | 0.108 | 0.097 | 0.148 |
| | Val | valine | 0.063 | 0.086 | 0.106 | 0.140 | 0.068 | 0.096 | 0.132 | 0.143 |
| | Met | methionine | 0.015 | 0.012 | 0.009 | 0.014 | 0.018 | 0.012 | 0.011 | 0.015 |
| | Cys | cysteine | 0.001 | 0.004 | 0.003 | 0.002 | 0.004 | 0.002 | 0.002 | 0.001 |
| | Ile | isoleucine | 0.021 | 0.024 | 0.032 | 0.029 | 0.027 | 0.027 | 0.025 | 0.038 |
| | Leu | leucine | 0.022 | 0.029 | 0.025 | 0.025 | 0.020 | 0.033 | 0.027 | 0.031 |
| | Phe | phenylalanine | 0.064 | 0.067 | 0.077 | 0.060 | 0.043 | 0.064 | 0.035 | 0.052 |
| | Trp | tryptophan | 0.072 | 0.150 | 0.107 | 0.170 | 0.083 | 0.070 | 0.058 | 0.050 |
| | Lys | lysine | 0.027 | 0.036 | 0.059 | 0.057 | 0.027 | 0.039 | 0.037 | 0.078 |
| | total | | 1.718 | 5.872 | 4.876 | 10.447 | 2.931 | 5.104 | 5.240 | 9.353 |

Note)
F₄ lines in Table 2 were all obtained by crossing Kyukei 305 with EnB1.

As is understood from Table 2, total free amino acid contents in the seeds of the F₄ line (No. 1) having all subunits of β-conglycinin and glycinin were at low levels. Moreover, it is also understood that causing genetic deficiency in any of the subunits of β-conglycinin and glycinin in soybean seeds has an effect of elevating free amino acid contents in soybean seeds by a certain level. Furthermore, it was more effective to cause genetic deficiency in any of the subunits of glycinin, in addition to causing genetic deficiency in the subunits of β-conglycinin. In particular, it was shown that not only the F₄ line (No. 16 in Table 2) genetically lacking all subunits of β-conglycinin and glycinin, but also the F₄ line (No. 12 in Table 2) genetically lacking groups I (A₁ₐB₂, A₂B₁ₐ and A_{1b}B_{1b}) and IIa (A₅A₄B₃ ) subunits of glycinin in addition to having a genetic deficiency in the subunits of β-conglycinin had the characteristics of a soybean having high levels of free amino acids. Among free amino acids, particularly significantly increased free amino acids were arginine, asparagine, histidine and glutamine.

It was shown again by this example, as well as Example 1, that a soybean genetically lacking all subunits of β-conglycinin and glycinin contains high levels of free amino acids. Furthermore, it was newly revealed by the example 2 that a soybean caused to be genetically deficient in all subunits of β-conglycinin and groups I (A₁ₐB₂, A₂B₁ₐ and A_{1b}B_{1b}) and IIa (A₅A₄B₃) subunits of glycinin, that is, a soybean having only subunit IIb (A₃B₄) of glycinin, is a soybean having high levels of free amino acids.

### Example 3

Next, to demonstrate that a soybean having high levels of free amino acids contains in a genetically stable manner high levels of free amino acids in the seeds thereof, the following experiment was conducted. Specifically, 3 QF2F₃ lines (QF2F₃-1, QF2F₃-2 and QF2F₃-3) genetically lacking all subunits of β-conglycinin and glycinin obtained in Example 1 were cultivated with conventional soybean varieties (Fukuyutaka and Tachiyutaka) having all subunits of β-conglycinin and glycinin, a conventional soybean variety (Enrei) genetically lacking only the A₅A₄B₃ subunit of glycinin, and β-conglycinin-deficient soybean (Kyukei 305) and glycinin-deficient soybean (EnB1) used as parents for crossing in outdoor general farmland (Nishi-goshi machi, Kumamoto, Japan; black volcanic ash soil) for 3 years from 1999 to 2001 (1999: with an average temperature of 23.8° C., average maximum temperature of 28.7° C., average minimum temperature of 19.5° C. and precipitation of 811.5 mm; 2000: with an average temperature of 24.3° C., average maximum temperature of 29.3° C., average minimum temperature of 19.7° C. and precipitation of 453.0 mm; 2001: with an average temperature of 24.1° C., average maximum temperature of 29.2° C., average minimum temperature of 19.4° C., and precipitation of 682.5 mm). Genetic fixation of agricultural properties was attempted via succeeding generations of the line lacking all subunits of β-conglycinin and glycinin. Thus, the seeds of F₆ line (QF2F₆-1, QF2F₆-2 and QF2F₆-3) genetically lacking all subunits of β-conglycinin and glycinin and the seeds of certain varieties or lines (Fukuyutaka, Tachiyutaka, Enrei, Kyukei 305 and EnB1) as controls for comparison were obtained.

The amino acid contents of the free amino acid extracts, which had been obtained similarly to the above Example 1 except for using the pulverized seeds of each soybean, were measured, and then total free amino acid content was calculated. Table 3 shows the results.

TABLE 3
Results-3 Analysis of free amino acids in soybean seeds

| | | | | Kyukei | | | | |
|---|---|---|---|---|---|---|---|---|
| Variety or line name | Fukuyutaka | Tachiyutaka | Enrei | 305 | EnB1 | QF2F₆-1 | QF2F₆-2 | QF2F₆-3 |

| | | | | | | | | | | |
|---|---|---|---|---|---|---|---|---|---|---|
| β-conglycinin | α | | + | + | + | − | + | − | − | − |
| subunit | α′ | | + | + | + | − | + | − | − | − |
| | β | | + | + | + | − | + | − | − | − |
| glycinin subunit | I | A₁ₐB₂, A₂B₁ₐ, A_{1b}B_{1b} | + | + | + | + | − | − | − | − |
| | IIa | A₅A₄B₃ | + | + | − | + | − | − | − | − |
| | IIb | A₃B₄ | + | + | + | + | − | − | − | − |
| free amino acid | Asp | aspartic acid | 0.297 | 0.293 | 0.294 | 0.521 | 0.282 | 0.824 | 1.055 | 1.022 |
| (mg/g seed) | Glu | glutamic acid | 0.238 | 0.400 | 0.389 | 0.253 | 0.331 | 0.790 | 1.030 | 1.264 |
| | Ser | serine | 0.024 | 0.025 | 0.049 | 0.021 | 0.015 | 0.055 | 0.064 | 0.107 |
| | Asn | asparagine | 0.056 | 0.111 | 0.428 | 0.060 | 0.213 | 1.048 | 2.301 | 1.872 |
| | Gly | glycine | 0.015 | 0.023 | 0.038 | 0.014 | 0.022 | 0.031 | 0.033 | 0.052 |
| | Gln | glutamine | 0.007 | 0.005 | 0.004 | 0.003 | 0.003 | 0.040 | 0.054 | 0.121 |
| | His | histidine | 0.024 | 0.068 | 0.172 | 0.018 | 0.091 | 0.261 | 0.331 | 0.413 |
| | Thr | threonine | 0.024 | 0.021 | 0.026 | 0.016 | 0.013 | 0.026 | 0.037 | 0.042 |
| | Ala | alanine | 0.054 | 0.087 | 0.152 | 0.060 | 0.104 | 0.204 | 0.286 | 0.407 |
| | Arg | arginine | 0.116 | 0.354 | 0.229 | 0.163 | 0.520 | 4.243 | 3.817 | 4.773 |
| | Pro | proline | 0.039 | 0.062 | 0.060 | 0.033 | 0.053 | 0.046 | 0.054 | 0.056 |
| | Tyr | tyrosine | 0.094 | 0.099 | 0.097 | 0.078 | 0.098 | 0.120 | 0.132 | 0.175 |
| | Val | valine | 0.038 | 0.048 | 0.055 | 0.040 | 0.044 | 0.061 | 0.099 | 0.064 |
| | Met | methionine | 0.038 | 0.051 | 0.059 | 0.036 | 0.039 | 0.033 | 0.035 | 0.024 |
| | Cys | cysteine | 0.001 | 0.001 | 0.001 | 0.001 | 0.001 | 0.001 | 0.000 | 0.000 |
| | Ile | isoleucine | 0.022 | 0.029 | 0.031 | 0.022 | 0.024 | 0.039 | 0.058 | 0.040 |
| | Leu | leucine | 0.023 | 0.030 | 0.043 | 0.025 | 0.024 | 0.029 | 0.038 | 0.038 |
| | Phe | phenylalanine | 0.055 | 0.061 | 0.059 | 0.049 | 0.068 | 0.062 | 0.079 | 0.090 |
| | Trp | tryptophan | 0.188 | 0.238 | 0.230 | 0.096 | 0.197 | 0.068 | 0.105 | 0.160 |
| | Lys | lysine | 0.023 | 0.026 | 0.020 | 0.020 | 0.026 | 0.041 | 0.045 | 0.062 |
| | total | | 1.377 | 2.033 | 2.437 | 1.530 | 2.167 | 8.022 | 9.655 | 10.784 |

Note 1)
Fukuyutaka, Tachiyutaka and Enrei are conventional soybean varieties.
Note 2)
Kyukei 305 and EnB1 are mother and father plants of QF2F₆-1, QF2F₆-2 and QF2F₆-3.

As is clear from Table 3, total free amino acid contents of soybean varieties (Fukuyutaka, Tachiyutaka and Enrei) vary in the range of 1.38 to 2.44 mg/g. In the case of Enrei (deficient in A₅A₄B₃ subunit of glycinin), which has the highest total free amino acid content, asparagine and histidine contents are particularly high. In contrast, in the case of the parents for crossing, Kyukei 305 and EnB1, total free amino acid contents are 1.53 and 2.17 mg/g, respectively, which are not so different from those of the conventional soybean varieties, but the arginine content of EnB1 is higher than those of the soybean varieties.

The total free amino acid contents of soybeans QF2F₆-1, QF2F₆-2 and QF2F₆-3 cultivated in general farmland ranged from 8.02 to 10.78 mg/g, which were below the half of that of QF2F₃ seeds (Table 1) obtained by cultivation within a glasshouse, but were clearly higher than those of the conventional soybean varieties (Fukuyutaka, Tachiyutaka and Enrei) and parents for crossing (Kyukei 305 and EnB1) cultivated under the same conditions. In particular, significantly increased free amino acids were arginine, asparagine, histidine and glutamine, agreeing with conditions regarding amino acids observed to increase in the soybeans having high levels of free amino acids as verified in Example 2.

Altogether, both F₃ seeds (see Example 1) and F₆ seeds (see Example 3) lacking major subunits of β-conglycinin and glycinin contained free amino acids at levels at least twice or approximately 3 to 5 times or more greater than those of the conventional soybean varieties. It shows genetically stable maintenance of the property of having high levels of free amino acids. Thus, it can be verified that a technique combining all types of deficiencies, including a deficiency in β-conglycinin and a deficiency in each subunit of glycinin is an effective technique to produce a soybean having high free amino acid content.

### Example 4

Next, it was verified that the soybean of the present invention having high levels of free amino acids contained free amino acids at high concentrations in the seeds when analyzed by a different method. QF2F₆-4 seeds genetically lacking all subunits of β-conglycinin and glycinin were sown in pots (filled with a mixture of culture soil and humus soil at a 2:1 ratio) within a heating glasshouse (Fukuyama-shi, Hiroshima, Japan; adjusted to an average temperature of 25° C., and having a natural day length) in December 2002, and then the seeds were obtained in March 2003. The seeds were pulverized, approximately 250 mg of the seeds was precisely weighed, and the resultant was then used in the determination of free amino acids. In addition, for comparison, a conventional soybean variety (Jack) having all subunits composing β-conglycinin and glycinin was cultivated under the same conditions employed for QF2F₆-4, and then the seeds were obtained. These seeds for comparison were also pulverized, approximately 250 mg of the seeds was precisely weighed, and the resultant was then used in the determination of free amino acids.

For the determination of free amino acids, 2 ml of 50 mM potassium phosphate buffer (pH 5.6) containing 8% TCA (trichloroacetic acid) was added to 250 mg of the pulverized seeds that had been precisely weighed as described above. After 1 hour of agitation at room temperature, centrifugation was performed at 10,000 g for 10 minutes, thereby obtaining the supernatant. The supernatant was filtered with a 0.45 mm filter. 10 ml of the filtered supernatant was analyzed using a high speed amino acid analyzer (model L-8500, Hitachi). The total sum was calculated to give total free amino acid content. Table 4 shows the results.

TABLE 4
Results-4 Analysis of free amino acids in soybean seeds

| Variety or line name | | | Jack | QF2F₆-4 |
|---|---|---|---|---|
| β-conglycinin subunit | α | | + | − |
| | α′ | | + | − |
| | β | | + | − |
| glycinin subunit | I | A₁ₐB₂, A₂B₁ₐ, A_{1b}B_{1b} | + | − |
| | IIa | A₅A₄B₃ | + | − |
| | IIb | A₃B₄ | + | − |
| free amino acid | Asp | aspartic acid | 0.385 | 1.512 |
| (mg/g seed) | Glu | glutamic acid | 0.295 | 0.959 |
| | Ser | serine | 0.015 | 0.021 |
| | Asn | asparagine | 0.112 | 2.885 |
| | Gly | glycine | 0.111 | 0.236 |
| | Gln | glutamine | ND | 0.144 |
| | His | histidine | 0.226 | 0.516 |
| | Thr | threonine | 0.017 | 0.046 |
| | Ala | alanine | 0.044 | 0.103 |
| | Arg | arginine | 0.486 | 28.385 |
| | Pro | proline | 0.015 | 0.022 |
| | Tyr | tyrosine | 0.007 | 0.017 |
| | Val | valine | 0.028 | 0.071 |
| | Met | methionine | 0.008 | 0.015 |
| | Cys | cysteine | 0.037 | 0.077 |
| | Ile | isoleucine | 0.014 | 0.044 |
| | Leu | leucine | 0.019 | 0.061 |
| | Phe | phenylalanine | 0.017 | 0.016 |
| | Trp | tryptophan | 0.141 | 0.040 |
| | Lys | lysine | 0.051 | 0.170 |
| | total | | 2.028 | 35.340 |

Note 1)
Jack is a conventional soybean variety.

Total free amino acid content in the seeds of the conventional soybean variety (Jack) having all subunits composing β-conglycinin and glycinin in Table 4 was 2.23 mg/g, which was at the same level as those of conventional soybean varieties (Fukuyutaka, Tachiyutaka and Enrei) and parents for crossing (Kyukei 305 and EnB1) that had been analyzed by different methods as shown in Table 3. However, total free amino acid content in the seeds of QF2F₆-4 genetically lacking all subunits of β-conglycinin and glycinin was 35.3 mg/g, which was 3 times or more greater than those of QF2F₆-1, QF2F₆-2 and QF2F₆-3 in Table 3. Furthermore, total free amino acid content in the seeds of QF2F₆-4 was 17 times or more greater than that of the conventional soybean variety (Jack) that had been cultivated under the same conditions as those used for QF2F₆-4. Furthermore, significantly increased free amino acids were arginine, asparagine, histidine and glutamine, agreeing with conditions regarding amino acids observed to increase in the soybeans having high levels of free amino acids as verified in Examples 2 and 3. In particular, argine was contained at a level 58 times or more greater than that of the conventional soybean variety (Jack).

Altogether, 2 different types of methods (Examples 3 and 4) for measuring amino acids and total free amino acid contents of the conventional soybean variety were almost equivalent to each other. However, the soybean seeds genetically lacking all subunits of β-conglycinin and glycinin contained free amino acids at levels approximately 3 to 17 times greater than those of the conventional soybean varieties. Thus, it was shown that even with different methods for analyzing amino acids, results indicating a property of high levels of free amino acids were maintained. Hence, it can be verified that a technique combining all types of deficiencies, including a deficiency in β-conglycinin and a deficiency in each subunit of glycinin, is an effective technique for producing a soybean having high free amino acid content.

### Example 5

The fact that causing genetic deficiency in all subunits of β-conglycinin is important to produce a soybean having high levels of free amino acids was verified using a conventional soybean variety genetically lacking most of β-conglycinin (hereinafter, referred to as a β-conglycinin-incompletely-deficient soybean), specifically, genetically lacking α and α′ subunits among the subunits of β-conglycinin and further having reduced β-subunit, as a parent for crossing, instead of Kyukei 305 (β-conglycinin subunit-completely-deficient soybean). Specifically, Yumeminori, the β-conglycinin-incompletely-deficient soybean, was crossed with EnB1 genetically lacking all subunits of glycinin, and then a β-conglycinin-incompletely-deficient and glycinin-deficient soybean (hereinafter, referred to as TF2) was obtained from the filial generation. The free amino acid contents were measured similarly to the method of Example 1.

This example was performed under the same conditions as, and simultaneously with, examples given in Examples 1 and 3. Specifically, the β-conglycinin-incompletely-deficient soybean (Yumeminori) and the glycinin-deficient soybean (EnB1) were grown by pot cultivation within a heating glasshouse during a period from December 1997 to March 1998 and then crossed, so that F₁ seeds were obtained. Next, the F₁ seeds were sown in general farmland in July 1998, and then the germinated F₁ plants were grown to maturing until the beginning of October 1998, so that F₂ seeds were obtained. The cotyledon portions (approximately 10 mg) of the obtained F₂ seeds were scraped off, and then the presence or the absence of each subunit group of β-conglycinin and glycinin was determined by the SDS-polyacrylamide gel electrophoresis method according to the method of Kitamura et al (1984). Thus, TF2 genetically lacking all subunits of glycinin and α and α′ subunits of the subunits of β-conglycinin and having reduced β-subunit was selected. Furthermore, the F₂ seeds were sown in pots within a heating glasshouse in December 1998, the germinated F₂ plants were grown, and then F₃ seeds were obtained in March 1999. For these F₃ seeds, the presence or the absence of each subunit of β-conglycinin and glycinin was determined again using approximately 10 grains of F₃ seeds by the SDS-polyacrylamide gel electrophoresis method. Thus, a β-conglycinin-incompletely-deficient and glycinin-deficient line, which was genetically deficient in all subunits of glycinin and α and α′ subunits of the subunits of β-conglycinin and had reduced β-subunit, was selected. The thus selected β-conglycinin-incompletely-deficient and glycinin-deficient line was cultivated together with Yumeminori, the β-conglycinin-incompletely-deficient variety, in a general farmland for 3 years from 1999 to 2001 under the same conditions as those given in the above Example 2, and then their seeds were obtained.

Using the pulverized seeds obtained from the above-selected TF2F₆-1 line and Yumeminori, the β-conglycinin-incompletely-deficient variety, total free amino acid contents were calculated by the method similar to that of the above Example 1. Table 5 shows the results.

TABLE 5
Results-5 Analysis of free amino acids in soybean seeds

| Variety or line name | | | Yumeminori | TF2F₆-1 |
|---|---|---|---|---|
| β-conglycinin | α | | − | − |
| subunit | α′ | | − | − |
| | reduced β | | + | + |
| glycinin subunit | I | | + | − |
| | IIa | | + | − |
| | IIb | | + | − |
| free amino acid | Asp | aspartic acid | 0.257 | 0.334 |
| (mg/g seed) | Glu | glutamic acid | 0.480 | 0.660 |
| | Ser | serine | 0.030 | 0.022 |
| | Asn | asparagine | 0.146 | 0.359 |
| | Gly | glycine | 0.026 | 0.025 |
| | Gln | glutamine | 0.011 | 0.006 |
| | His | histidine | 0.056 | 0.107 |
| | Thr | threonine | 0.021 | 0.019 |
| | Ala | alanine | 0.091 | 0.155 |
| | Arg | arginine | 0.393 | 1.150 |
| | Pro | proline | 0.053 | 0.061 |
| | Tyr | tyrosine | 0.095 | 0.112 |
| | Val | valine | 0.047 | 0.055 |
| | Met | methionine | 0.056 | 0.035 |
| | Cys | cysteine | 0.001 | 0.000 |
| | Ile | isoleucine | 0.025 | 0.029 |
| | Leu | leucine | 0.028 | 0.029 |
| | Phe | phenylalanine | 0.063 | 0.075 |
| | Trp | tryptophan | 0.162 | 0.152 |
| | Lys | lysine | 0.030 | 0.028 |
| | total | | 2.071 | 3.411 |

Note 1)
Yumeminori is a conventional soybean variety and mother plant of TF2F₆-1. The father plant of TF2F₆-1 is EnB1.
Note 2)
Production conditions for the seeds of Yumeminori and TF2F₆-1 are the same as those employed for the plants shown in Table 3.

As is clear from Table 5, the total free amino acid content of Yumeminori, the β-conglycinin-incompletely-deficient variety, was 2.07 mg/g, which was not so different from those of conventional soybean varieties cultivated under the same conditions (Table 3). However, the glutamic acid and arginine contents of Yumeminori were somewhat higher than those of the conventional soybean varieties (Table 3).

Furthermore, the total free amino acid content of TF2F₆-1 line is somewhat higher than those of the conventional soybean varieties (Table 3) cultivated under the same conditions. However, the increased level thereof is clearly less than those of the 3 lines (QF2F₆-1, QF2QF₆-2 and QF2F₆-3) (see Table 3) obtained in Example 3.

By the above example, it can be verified that when a soybean is caused to be genetically deficient in all subunits of glycinin and in most of β-conglycinin, but contains some of the subunits of β-conglycinin, the soybean does not come to have high free amino acid content

### Example 6

Next, it was verified that a soybean genetically lacking all subunits of β-conglycinin and glycinin can be produced using another combination for crossing other than that of Kyukei 305 genetically lacking each subunit of β-conglycinin and EnB1 genetically lacking each subunit of glycinin as shown in the above Examples 1, 2 and 3. Specifically, it was demonstrated that a soybean genetically lacking all subunits of β-conglycinin and glycinin can also be produced by crossing the soybean QF2F₃-1 genetically lacking all subunits of β-conglycinin and glycinin obtained in the above Example 3 with Fukuyutaka, which is a conventional soybean variety having all subunits of β-conglycinin and glycinin.

In February 2001, QF2F₃-1 seeds and Fukuyutaka seeds were sown in pots (filled with black volcanic ash soil) in a heating glasshouse (Nishi-goshi machi, Kumamoto, Japan; adjusted to a minimum temperature of 22° C. and a maximum temperature of 32° C.; during 1 month after sowing, lightning was performed everyday with fluorescent lamps twice a day (4 p.m. to 9 p.m. and 3 a.m. to 8 a.m. in the next morning) to give a day length of 18 hours. After this period, plants were grown under natural day length.). The plants were grown for approximately 40 to 45 days, crossed, and then 15 grains of the F₁ seeds were sampled in May 2001. 15 grains of the obtained F₁ seeds were sown in general farmland (Nishi-goshi machi, Kumamoto, Japan, black volcanic ash soil) in July 2001, and then the F₂ seeds were obtained in October 2001 (with an average temperature of 24.1° C., average maximum temperature of 29.2° C., average minimum temperature of 19.4° C. and precipitation of 682.5 mm). For 405 grains of the F₂ seeds, the presence or the absence of each subunit of β-conglycinin and glycinin was measured by the SDS-polyacrylamide gel electrophoresis method. Thus, 4 grains of F₂ seeds genetically lacking all subunits of β-conglycinin and glycinin could be selected. Based on this result, it was shown that a soybean genetically lacking all subunits of β-conglycinin and glycinin could also be produced by crossing a soybean genetically lacking all subunits of β-conglycinin and glycinin with a soybean having all subunits of β-conglycinin and glycinin.

### Example 7

Furthermore, it was verified that the soybean of the present invention having high levels of free amino acids can normally grow in outdoor general farmland and can produce normal seeds without causing any actual problems. 3 lines (QF2F₅-1, QF2F₅-2 and QF2F₅-3) and conventional soybean varieties (Fukuyutaka, Enrei and Tachiyutaka) cultivated in outdoor general farmland in 2001 in the aforementioned example, and β-conglycinin-deficient soybean (Kyukei 305) and glycinin-deficient soybean (EnB1) that had been crossed as parents of the above lines were examined.

The day on which more than half of the plants of each of the above lines, which had been cultivated in outdoor general farmland, began flowering was determined as the flowering time, and the day on which 80% or more of the same reached maturity was determined as the maturity time. At the maturity time, 20 stocks were sampled, and then the main stem length, the number of main stem nodes, the number of branches, and the weight of 100 grains and the seed weight were measured according to description given in the investigation report on the classification of specific characters of seeds and stocks (Shubyo Tokusei Bunrui Cho-sa Ho-koku-sho, Japan Speciality Agriculture Products Association, March 1995). Table 6 shows the results.

TABLE 6
Basic agricultural properties of soybean having high levels of free amino acids

| Variety or line name | Fukuyutaka | Tachiyutaka | Enrei | Kyukei 305 | EnB1 | QF2F₅-1 | QF2F₅-2 | QF2F₅-3 |
|---|---|---|---|---|---|---|---|---|
| Presence or absence | + | + | + | − | + | − | − | − |
| of β-conglycinin | | | | | | | | |
| Presence or absence | + | + | +* | + | − | − | − | − |
| of glycinin | | | | | | | | |
| Flowering time | August 18 | August 12 | August 10 | August 18 | August 11 | August 11 | August 13 | August 14 |
| Maturity time | October 31 | October 18 | October 15 | October 31 | October 15 | October 15 | October 22 | October 22 |
| Main stem length | 58.0 | 37.7 | 38.7 | 58.1 | 41.1 | 30.5 | 35.7 | 39.0 |
| Number of main | 15.6 | 13.1 | 11.8 | 16.1 | 13.1 | 12.4 | 12.2 | 14.0 |
| stem nodes | | | | | | | | |
| Number of branches | 3.9 | 3.7 | 3.9 | 4.8 | 3.6 | 4.0 | 3.5 | 3.6 |
| 100 grains weight | 32.2 | 30.2 | 36.1 | 31.2 | 20.1 | 19.6 | 22.7 | 22.9 |
| (15% moisture | | | | | | | | |
| contents) | | | | | | | | |
| grain yield kg/a | 40.9 | 35.4 | 30.3 | 43.0 | 30.4 | 28.9 | 28.8 | 31.3 |

Note 1)
Fukuyutaka, Tachiyutaka and Enrei are conventional soybean varieties.
Note 2)
Enrei lacks group IIa subunit of glycinin.
Note 3)
Plant bodies that had produced seeds shown in Table 3 were examined.

As is clear from Table 6, the flowering time and the maturity time of QF2F₅-1 are almost the same as those of EnB1, but come considerably earlier than those of Kyukei 305, the other parent for crossing. In contrast, the flowering time and the maturity time of QF2F₅-2 and QF2F₅-3 come somewhat later, which is an intermediate date between the date of EnB1 and that of Kyukei 305. Regarding main stem length among characteristics concerning vegetative growth, any lines having high levels of free amino acids are shorter than parents for crossing. However, regarding the number of main stem nodes, that of QF2F₅-3 is somewhat greater than that of EnB1. Regarding the number of branches, any lines having high levels of free amino acids have fewer branches than Kyukei 305, but somewhat greater number of these lines have almost the same number of branches as that of EnB1. When compared with soybean varieties regarding characteristics other than main stem length, the soybean lines having high levels of free amino acids are not greatly inferior to Enrei or Tachiyutaka, whose flowering times and maturity times were close to those of the soybean lines.

Regarding the weight of 100 grains representing seed size, those of QF2F₅-1, QF2F₅-2 and QF2F₅-3 are all less than that of Kyukei 305. However, when compared with that of EnB1, those of QF2F₅-2 and QF2F₅-3 are somewhat greater. Also regarding the seed weight, those of QF2F₅-1, QF2F₅-2 and QF2F₅-3 were all clearly less than that of Kyukei 305, but are not so different from that of EnB1. When compared with conventional soybean varieties, the soybean weights of QF2F₅-1, QF2F₅-2 and QF2F₅-3 are all intermediate values between those of Enrei or Tachiyutaka, whose flowering time and maturity time were close to those thereof. Furthermore, the total free amino acid content of QF2F₆-3 seed obtained from QF2F₅-3, with the highest seed weight, was the highest among the three lines (see Table 3).

As described above, the general agricultural properties of 3 lines, QF2F₅-1, QF2F₅-2 and QF2F₅-3, are not greatly inferior to those of conventional soybean varieties whose flowering time and maturity time are close to those thereof, so that they can be said to be soybean lines that can be applied for agricultural use without causing any problems.

Cited References

Nielsen, N. C., Dickinson, C. D., Cho, T. -J Thanh, V. H., Scallon, B. J., Fischeer, R. L., Sims, T. L., Drews, G. N. and Goldberg. 1989. Characterization of the glycinin geno family in soybean. Plant Cell. 1: 313-328.
Ogawa, T. Tayama, E., Kitamura, K. and Kaizuma, N. 1989. Genetic improvement of seed storage proteins using three variants alleles of 7S globulin subunits in soybean (Glycine max L.) Jpn. J. Breed. 39: 137-147.
Yagasaki, K., Kaizuma, K. and Kitamura, K. 1996. Inheritance of glycinin molecules lacking the subunits in soybean (Glycine max (L.) Merr.). Breeding Sci. 46: 11-15.
Yagasaki, K., Kousaka, F. and Kitamura, K. 2000. Potential improvement of soymilk gelation properties by using soybeans with modified protein subunit compositions. Breed. Sci. 50: 101-107.
Hajika, M., Takahashi, M., Sakai, S. and Igita, K. 1996. A new genotype of a 7S globulin (β-conglycinin) detected in wild soybeans (Glycine soja Sieb. et. Zucc.). Jpn J. Breed. 46: 385-386.
Hajika, M., Takahashi, M., Sakai, S. and Matsunaga., R 1998. Dominant inheritance of a trait lacking β-conglycinin detected in a wild soybean line. Breed. Sci.48: 383-386.
Takahashi, M., Hajika, M., Matsunaga, R. Komatsu, K., Obata, A. and Kanegai, R2000. Breeding of soybean variety lacking β-conglycinin by the introduction of Scg gene from wild soybean. Proceedings of the Third International Soybean Processing and Utilization Conference. pp. 45-46.
Yoshihiko Uematsu, Makita Hajika, Masakazu Takahashi, Kazuhiro Yagasaki, Masayoshi Teraishi, Masaru Tamba and Masao Ishimoto. 1999. Protein Content and Composition in 7S and 11S globulin-deficient soybean line. Breeding Research 1 (Supplement 1): 154
Kitamura, K. 1984. Biochemical characterization of lipoxygenase lacking mutant, L-1less, L-2 less and L-3 less soybean. Agric. Biol. Chem. 48: 2339-2346.
Park, K. G. M., Hays, P. D., Garlick, P. J., Swell, H., and Eremin, O. 1991. Stimulation of lymphocyte natural cytotoxicity by L-arginine. The Lancet 337: 645-646.
Kirk S. J., Hurson M., Regan M. C. 1993. Arginine stimulates wound healing and immune function in elderly human beings. Surgery 114: 155-60.
Kreider R. B., Miriel V., and Bertun. E. 1993. Amino acid supplementation and exercise performance: proposed ergogenic value. Sports Medicine 16: 190-209.
Rennie, M. J. 1996. Glutamine metabolism and transport in skeletal muscle and heart and their clinical relevance. Journal of Nutrition 126(4): 1142-1149.
Newsholme, E. A. and Calder P. C. 1997. The proposed role of glutamine in some cells of the immune system and speculative consequences for the whole animal. Nutrition 13: 728-730.

1. A soybean, having total free amino acid content in the seed thereof that is higher than the content in the seeds of any of Fukuyutaka and Tachiyutaka having all subunits of β-conglycinin and glycinin, Enrei lacking only A₅A₄B₃ subunit of glycinin, Kyukei 305 lacking all subunits of β-conglycinin and EnB1 lacking all subunits of glycinin that are cultivated under similar conditions.

2. The soybean of claim 1, having total free amino acid content in the seed thereof that is at least 2 times or more greater than the content in the seeds of any of Fukuyutaka and Tachiyutaka having all subunits of β-conglycinin and glycinin, Enrei lacking only A₅A₄B₃ subunit of glycinin, Kyukei 305 lacking all subunits of β-conglycinin, and EnB1 lacking all subunits of glycinin that are cultivated under similar conditions.

3. The soybean of claim 1, wherein the total free amino acid content in the seed thereof is 8 mg or more per gram dry weight of the seed.

4. The soybean of claim 1, wherein the content of at least one of free amino acids selected from the group consisting of alginine, asparagine, histidine and glutamine among each of free amino acids contained in the seed is greater than that contained in the seed of any of Fukuyutaka and Tachiyutaka having all subunits of β-conglycinin and glycinin, Enrei lacking only A₅A₄B₃ subunit of glycinin, Kyukei 305 lacking all subunits of β-conglycinin and EnB1 lacking all subunits of glycinin that are cultivated under similar conditions.

5. The soybean of claim 4, wherein the contents of each free amino acids of arginine, asparagine, histidine and glutamine among each of free amino acids contained in the seed are each greater than the contents thereof in the seeds of any of Fukuyutaka and Tachiyutaka having all subunits of β-conglycinin and glycinin, Enrei lacking only A₅A₄B₃ subunit of glycinin, Kyukei 305 lacking all subunits of β-conglycinin and EnB1 lacking all subunits of glycinin wherein they are cultivated under similar conditions.

6. The soybean of claim 1, genetically lacking at least α, α′ and β subunits of β-conglycinin, and A₁ₐB₂, A₂B₁ₐ, A_{1b}B_{1b} and A₅A₄B₃ subunits of glycinin.

7. The soybean of claim 6, genetically lacking all subunits of β-conglycinin and glycinin.

8. A method of producing the soybean of claim 6, comprising either step of crossing a soybean lacking one or more subunits selected from the group consisting of α, α′ and β subunits of β-conglycinin, and A₁ₐB₂, A₂B₁ₐ, A_{1b}B_{1b} and A₅A₄B₃ subunits of glycinin with a soybean lacking all the subunits contained in the above soybean among the subunits in the above group, or step of crossing a soybean lacking all of the above subunits with a soybean having all of or some of these subunits, wherein at least one of the two soybeans to be crossed herein has the A₃B₄ subunit of glycinin.

9. The method of producing the soybean of claim 7, comprising either step of crossing a soybean lacking one or more subunits selected from the group consisting of α, α′ and β subunits of β-conglycinin and A₁ₐB₂, A₂B₁ₐ, A_{1b}B_{1b}, A₅A₄B₃ and A₃B₄ subunits of glycinin with a soybean lacking all the subunits of the soybean among the above subunits, or step of crossing a soybean lacking all of the subunits in the above group with a soybean having all of or some of these subunits.

10. The method of claim 8 or 9, comprising a step of crossing Kyukei 305 lacking all subunits of β-conglycinin with EnB1 lacking all subunits of glycinin.

11. The method of any one of claims 8 to 10, further comprising a step of selecting a line having only the A₃B₄ subunit of glycinin among the subunits of β-conglycinin and glycinin, or a line lacking all subunits of β-conglycinin and glycinin following the step of crossing.

12. A functional food, which is produced using the soybean seed of any one of claims 1 to 7 as a raw material, and wherein the total free amino acid content is increased.

13. A method of producing a functional food wherein the total amino acid content is increased, using the soybean seed of any one of claims 1 to 7 as a raw material.
